(19) United States
(12) Patent Application Publication
(10) Pub. No.: US 2026/0224155 A1
(43) Pub. Date: Aug. 6, 2026

(54) CARDIAC RHYTHM MONITORING

(71) Applicant: CoreMap, Inc., Burlington, MA (US)

(72) Inventor: Peter S. Spector, Burlington, MA (US)

(21) Appl. No.: 19/530,702

(22) Filed: Feb. 5, 2026

Related U.S. Application Data
(60) Provisional application No. 63/754,899, filed on Feb. 6, 2025.

(51) Int. Cl. A61B 5/361; A61B 5/00; A61B 5/287; A61B 5/343; A61B 5/367

(52) U.S. Cl. CPC A61B 5/361; A61B 5/287; A61B 5/343; A61B 5/367; A61B 5/6852; A61B 2560/0295; A61B 2562/046

(57) ABSTRACT

Methods include obtaining measurements of electrical activity of a heart, organizing the measurements into one group with values within a first range and a second group with values within a second range, and associating the first group with a first incidence of fibrillation and the second group with a second incidence of fibrillation in the heart. The values may comprise cycle lengths or median cycle lengths. Methods may include grouping the measurements by the median cycle lengths and a second value such as a direction of propagation. The first incidence of atrial fibrillation may comprise electrical electrophysiological activity resulting from a first focal origin or multiple reentrant circuit and the second incidence of atrial fibrillation may comprise electrophysiological activity resulting from a second focal origin or multiple reentrant circuit, potentially independent from the first focal origin or multiple reentrant circuit.

### BACKGROUND

Atrial fibrillation is the irregular activity of muscle fibers in the heart resulting in an arrhythmia associated with increased mortality and risk of stroke. Atrial fibrillation is a progressive disease that, if left untreated, may worsen over time, and can lead to various problems such as blood clots, stroke or heart failure. Mechanisms of atrial fibrillation are discussed in Roney, 2020, Challenges associated with interpreting mechanisms of AF, Arrhythmia & Electrophysiology Rev 8(4):273-284, the contents of which are incorporated by reference for all purposes.

Existing treatments for fibrillation include medication and surgical procedures intended to restore normal electrical activity of the atria. Medication is not always effective. When medication is not effective, clinicians may try ablation, a procedure that typically uses heat or cold energy to deliberately scar tissue of the heart, creating non-conducting regions of cardiac tissue that limit the irregular activity.

Unfortunately, ablation is also not always successful. It has been estimated that long-term success rates of ablation procedures may stand between 50 and 80%. See Calkins, 2009, Treatment of atrial fibrillation with antiarrhythmic drugs or radiofrequency ablation: two systematic literature reviews and meta-analyses, Circ Arrhythm Electrophysiol 2(4):349-6, incorporated by reference. Ablation is challenging due to difficulties in identifying specific locations at which to ablate in cardiac tissue.

Another issue is that in some cases of persistent atrial fibrillation, excitation patterns vary over time, a phenomenon that complicates mapping of activity in the cardiac substrate. Substrate mapping techniques to guide ablation may involve mapping the cardiac substrate to identify regions associated with activity that is arrhythmic. However, if the patterns of excitation vary, interpreting the map may be difficult. Accordingly, there is a need in the art for improved techniques for accurately mapping atrial fibrillation.

### SUMMARY

The invention provides systems and methods for analyzing electrophysiological activity associated with distinct and potentially independent incidents of atrial fibrillation in a single patient. Methods of the invention are implemented by obtaining a plurality of electrophysiological measurements. Subsets of those measurements are grouped by selected common elements. For example, beat cycle length may be determined and measurements from different segments in time may be assigned to first or second (or more) groups based on common characteristic cycle lengths.

Certain embodiments of the invention utilize median cycle lengths calculated within a sliding window over time. Using the groups, distinct incidents of atrial fibrillation are identified. When the groups are defined along a first dimension, such as cycle length median, methods may include obtaining measurements of a second dimension, such as direction of wave propagation in the cardiac substrate. Each subgroup may include sets of beats grouped by two-dimensions of measurements, such as cycle length median and direction values. On such a basis, the distinct incidents of atrial fibrillation that are identified in the subgroups result from potentially independent electrophysiological sources of atrial fibrillation, such as separate focal drivers or multi-wavelet re-entry circuits. Thus, the invention provides tools for separately mapping potentially independent but overlapping loci of atrial fibrillation on the same cardiac tissue.

Not only do methods and systems of the invention classify and document distinct and potentially independent regimes of atrial fibrillation, but methods of the invention also provide separate maps that show those independent regimes mapped onto a representation of the heart. As additional data are collected, e.g., during a cardiac mapping procedure, the incoming new data may be classified using systems and methods herein as being of one group or another and added to the appropriate respective map. The maps may be color coded or annotated to guide a clinician to a region of interest in the heart or to indicate which regime of atrial fibrillation is likely linked to certain identified data. The clinician may use those maps to guide a mapping or ablation procedure. Because the maps show the distinct incidences of atrial fibrillation appropriately segregated onto the corresponding map, the guidance is more accurate and the procedure has an improved chance of success, compared to a procedure performed using an unsegregated map.

In related embodiments, the invention provides systems and methods for analyzing cardiac activity to identify when a source (e.g., a driver) of cardiac fibrillation starts and stops in real time, from anywhere in the heart. Systems and methods of the invention measure cardiac signals using electrodes located within the heart or on the surface of the skin. A computer system coupled to the electrodes analyzes the cardiac signals as they are recorded to detect when a driver becomes active (the start time) and when the driver becomes inactive (the stop time). The systems and methods described provide a more complete and accurate view of drivers of cardiac fibrillation.

The identification of a start and/or stop time of a driver gives a unique insight into the activity of the driver and its effects on the heart. For example, the start and stop time of a driver provides a time period for when the driver was active. Additional cardiac signals that were recorded during that time period may be attributed to those active drivers and analyzed to determine the effect a driver has on other areas of the heart.

The cardiac signals and drivers may also be displayed on a cardiac map (such as a 3D representation of the heart) that may be segmented based on the time period a driver was active. In situations where more than one driver is present, cardiac signals may be categorized according to the drivers that were active during a chosen time period. From here, the categorized signals and corresponding drivers may be assigned to different cardiac maps based on various combinations of active drivers. The result is one or more cardiac maps that provide a visual representation of the electrical activity associated with drivers of cardiac fibrillation at different times. Such cardiac maps may also give information regarding intermittent or missed drivers if, for example, a cardiac map shows no driver or a different driver in a different location.

To provide additional measurements of cardiac signals, systems and methods of the invention may use an electrocardiogram (ECG). A traditional 12-lead ECG has the leads placed on the body in such a way that optimizes the recording of ventricular signals. However, systems and methods of the invention may place the leads in alternative locations. These alternative placements may increase sensitivity to P waves and optimize detection of atrial signals. The placements may also aid in determining which chamber of the heart a driver of atrial fibrillation is located in. One possible placement involves placing the V2, V4, and V6 leads on a patient's back. Frequency domain analysis of the resulting ECG may be performed to determine the number of drivers in a chamber of the heart.

In certain aspects, the invention provides methods of analyzing cardiac rhythm. Methods include obtaining measurements of electrical activity of a heart, organizing the measurements into one group with values within a first range and a second group with values within a second range, and associating the first group with a first incidence of fibrillation and the second group with a second incidence of fibrillation in the heart. The values may comprise cycle lengths or median cycle lengths. Methods may include grouping the measurements by the median cycle lengths and a second value such as a direction of propagation. The first incidence of atrial fibrillation may comprise electrical electrophysiological activity resulting from a first focal origin or multiple reentrant circuit and the second incidence of atrial fibrillation may comprise electrophysiological activity resulting from a second focal origin or multiple reentrant circuit, potentially independent from the first focal origin or multiple reentrant circuit.

Methods may include using the first and second groups to assign subsequent measurements of cardiac activity to the first or second incidence of fibrillation. Methods may include creating a first map of wave propagation of the first incidence of fibrillation and a second map of wave propagation of the second incidence of fibrillation. Methods may include displaying at least the first map or the second map on a display device of a computer system during a surgical procedure for ablation.

In some embodiments, obtaining measurements of cardiac electrical activity comprises placing a catheter comprising electrodes into a coronary sinus and creating a record of cycle length over a time between from about a minute to about an hour. The groupings of the data represent contributions from distinct electrophysiological phenomena. For example, the first incidence and the second incidence may be manifestations of independent physiological causes of atrial fibrillation. Each of the independent physiological causes may comprise a focal driver or multi-wavelet re-entry circuit.

In other aspects, the invention provides systems for analyzing cardiac rhythm. Preferred systems include a cardiac mapping catheter comprising electrodes operable to obtain measurements of electrical activity of a heart and a computer system communicably coupled to the cardiac mapping catheter. The computer system has at least once processor coupled to memory having instructions stored therein executable by the processor to cause the computer system to organize the measurements into one group with values within a first range and a second group with values within a second range and associate the first group with a first incidence of fibrillation and the second group with a second incidence of fibrillation in the heart. The values may include cycle lengths or median cycle lengths. In certain embodiments, the system groups the measurements by the median cycle lengths and a second value such as a direction of propagation.

The computer system may use the first and second groups to assign subsequent measurements to the first or second incidence of fibrillation. The computer system may be operable to create a first map of wave propagation of the first incidence of fibrillation and a second map of wave propagation of the second incidence of fibrillation. Systems may include a display device in a surgical theater operable to display at least the first map or the second map.

In certain embodiments, the computer system stores the first group and the second group in the memory and provides a report using the first group and second group to show the first incidence and the second incidence as manifestations of independent physiological causes of atrial fibrillation. The computer system may be operable to annotate the first map and the second map to show each of the independent physiological causes as a focal driver or multi-wavelet re-entry circuit. In some embodiments, the cardiac mapping catheter comprises an extended catheter body dimension for percutaneous insertion into a chamber of the heart, and a deformable head on a distal portion of the catheter body, with the electrodes disposed in an array over an area of the deformable head.

Other aspects of the invention provide methods for mapping atrial fibrillation. Exemplary methods include recording cardiac electrograms using a plurality of electrodes disposed on a distal portion of a mapping catheter and storing the electrograms in computer memory. Methods further include grouping, by the computer system, portions of the electrograms into at least two groups based at least on cycle length values determined from the electrograms and identifying an independent regime of atrial fibrillation in the heart for each of the at least two groups. The electrodes may be disposed in an array over an area or length of a deformable head at a distal portion of the mapping catheter. Further methods may include determining, from the electrograms and from time data for signals measured at the electrodes in the array over the area, at least one component of a direction of waves in the heart. In certain embodiments, the grouping is further based on the cycle length values and the component of the direction of the waves. The cycle length values may include median cycle length within a sliding window over different portion of the electrograms. Methods of the invention may include creating a total electrophysiological (EP) map (e.g., a non-segregated map) showing electrophysiological activity in heart and using the at least two groups to create segregated maps, e.g., at least a first map showing activity of a first regime of AF in the heart and a second map showing activity of a second regime of AF in the heart. Additional methods may include annotating (by operation of the computer system) at least the first map to show a region of a focal drive or multi-wavelet reentry causative of the first regime of AF. In an alternative embodiment, methods may include displaying at least the first map during a cardiac mapping procedure to show areas in the heart where additional data should be collected. In other embodiments, the computer system uses a sliding window over a time dimension of the electrogram to automatically determine cycle length medians within the sliding window and automatically group the portions of the electrograms into the two groups while the electrograms are being record. The time dimension may include at least about five minutes of dwell time during which the plurality of electrodes perform the recording in cardiac tissue and optionally the sliding window has a width between about 1 second and about 10 seconds in the time dimension (e.g., 2 seconds or 5 seconds). Methods may include identifying beats in the electrograms and assigning, to the at least two groups, sets of the beats based on directionality and cycle length. Related aspects provide a system comprising the mapping catheter and a computer system, in which the computer system has at least one processor coupled to a memory subsystem have instructions therein executable by the process to cause the computer system to perform the above-recited recording, grouping, and identifying steps.

In certain aspects, the invention provides a system for analyzing cardiac signals. The system may include an intracardiac catheter with electrodes on a distal portion of the catheter operable to measure cardiac signals of a heart, and a computer system coupled to the intracardiac catheter. The computer system is operable to analyze the measured cardiac signals and identify a start time or a stop time of at least one driver of atrial fibrillation in real time. In some embodiments, the computer system identifies the start time or stop time while the electrodes are measuring the cardiac signals. In certain embodiments, the electrodes may be positioned a distance away from at least one driver of atrial fibrillation and the computer system may still be operable to identify the start time or stop time of the driver.

The computer system may be operable to create a cardiac map and to segment the cardiac map based on a time period during which at least one driver was active. Where the at least one driver comprises at least two drivers, the computer system may be operable to categorize the cardiac signals based on the drivers that were active during the time period. The computer system may be operable to map the categorized cardiac signals on separate cardiac maps. In such embodiments, the separate cardiac maps may each represent a different combination of drivers that were active during the time period. The computer system may further identify the presence of an intermittent driver if a cardiac map displays no drivers or displays a different driver in a different location.

In additional embodiments, the computer system may be coupled to a 12-lead ECG. The 12-lead ECG may be optimized to detect atrial signals by placing leads V2, V4, and V6 on a patient's back. The computer system may be operable to identify a chamber of the heart that at least one driver is located in. Where the at least one driver comprises at least two drivers, the computer system may be operable to identify a chamber of the heart where each driver is located and to determine how many drivers are present in each chamber of the heart. In certain embodiments, the computer system determines how many drivers are in each chamber of the heart based on number of peaks in the frequency domain using the 12-lead ECG. The computer system may determine how many drivers are in each chamber of the heart based on relative amplitude of specific frequency peaks on different leads of the 12-lead ECG. The computer system may be operable to determine how much each driver affects electrical activity in the chamber each driver is located in. In some embodiments, the computer system is operable to determine a rate of an individual driver. The computer system may also be operable to determine variability of an individual driver.

Other aspects of the invention provide methods for analyzing cardiac signals. The method may include measuring cardiac signals from a heart using electrodes on a cardiac catheter and analyzing, with a computer system, the measured cardiac signals to identify a start time or a stop time of at least one driver of cardiac fibrillation in real time. The electrodes may be positioned a distance away from at least one driver of cardiac fibrillation and the computer system may still identify the start time or stop time of the driver.

Methods further include creating a cardiac map. In certain embodiments, the method may comprise segmenting the cardiac map based on a time period at least one driver was active. The at least one driver may comprise at least two drivers. Methods may include categorizing the cardiac signals based on the drivers that were active during the time period. Methods may further comprise mapping the categorized cardiac signals on separate cardiac maps. The separate cardiac maps may each represent a different combination of drivers that were active during the time period. Additional methods may include identifying presence of an intermittent driver if the cardiac map displays no drivers or displays a different driver in a different location. In some embodiments, the computer system is coupled to a 12-lead ECG. The 12-lead ECG may be optimized to detect atrial signals by placing leads V2, V4, and V6 on a patient's back. Methods may further comprise identifying a chamber of the heart that at least one driver is located in. The at least one driver may comprise at least two drivers. Methods may include identifying a chamber of the heart where each driver is located in. Methods may further include determining how many drivers are in each chamber of the heart. Determining how many drivers are in each chamber of the heart may be based on the number of peaks on a frequency domain using the 12-lead ECG. Determining how many drivers are in each chamber of the heart may be based on relative amplitude of specific frequency peaks on different leads of the 12-lead ECG. Methods may include determining how much each driver affects electrical activity in the chamber each driver is located in. The methods may further include determining a rate of an individual driver. In related embodiments, methods may include determining variability of an individual driver.

### BRIEF DESCRIPTION OF THE DRAWINGS

FIG. 1 shows a system for monitoring cardiac rhythm.

FIG. 2 shows steps of a method of analyzing cardiac rhythm.

FIG. 3 shows obtaining the measurements of the electrical activity of a heart.

FIG. 4 shows a graph of cycle length (CL) over time during atrial fibrillation (AF).

FIG. 5 shows a catheter head carrying an electrode array.

FIG. 6 shows a measurement of directionality of waves.

FIG. 7 is a plot of coronary sinus (CS) cycle length over time.

FIG. 8 displays the direction of activation along a CS catheter vs CS cycle length.

FIG. 9 shows a non-segregated map of electrophysiological activity in a heart.

FIG. 10 shows a first map showing CL median of a first regime of AF in the heart.

FIG. 11 shows a second map showing CL median of a second regime of AF in the heart.

FIG. 12 shows an unsegregated map of cycle length variance on a 3D model of a heart.

FIG. 13 shows a first map of cycle length variance for a first regime of AF.

FIG. 14 shows a second map of cycle length variance for second regime of AF.

FIG. 15 shows an unsegregated prominence map.

FIG. 16 shows a first prominence map for a first regime of AF.

FIG. 17 shows a second prominence map for a second regime of AF.

### DETAILED DESCRIPTION

The invention relates to methods and systems useful as a cardiac rhythm monitor. Systems and methods of the invention use cardiac mapping catheters to make electrophysiological measurements from cardiac tissue over time and process those measurements (typically electrograms) in a manner that accommodates changes in cardiac tissue that occur during the time of mapping. Systems of the invention analyze incoming measurements to identify stable regimes of behavior as well as changes from one stable regime of behavior (or atrial fibrillation) to a second regime of atrial fibrillation. Systems of the invention may use components of electrogram measurements, such as cycle length, to identify segments of the electrogram (e.g., beats) that fall into one group or another where each group is a manifestation of a potentially independent regime of atrial fibrillation.

Among other things, the invention makes use of the insight that a heart may exhibit more than one potentially-independent manifestation of atrial fibrillation. Each of those independent manifestations of atrial fibrillation may be referred to a “regime” or incidence of atrial fibrillation. Each incidence of atrial fibrillation may be associated with its own (potentially) independent electrophysiological cause, such as its own focal driver or multi-wavelet re-entry circuit. However, two apparently distinct incidences of atrial fibrillation, each with its own characteristic cycle length, locus, or direction, could ultimately share common causality but nevertheless manifest as electrograms from a mapping catheter that have distinct measurement characteristics. The present invention analyzes the electrograms and groups, or segregates, sub-segments of those electrograms into potentially independent regimes or incidences of atrial fibrillation. Having identified those regimes, subsequent measurements may be assigned, based on, e.g., a cycle length measurement, to the physiologically relevant regime of atrial fibrillation. An “incidence” of atrial fibrillation is a period of time during which there is a particular distribution of drivers of atrial fibrillation.

Methods of the invention have particular utility when mapping cardiac activity including, for example, when generating a viewable map with a representation of a heart with a graphic depiction of cardiac activity in the heart. Without segregation into the groups per the invention, all electrophysiological activity would be “drawn” on one map of the heart, even when all of that activity included manifestations from multiple, potentially independent incidences of atrial fibrillation. Using methods of the invention, those potentially independent incidences of atrial fibrillation are identified and the associated measurements may be added to, or represented by, their own separate maps. Thus, a map of all activity may be segregated or separated to create a first map showing activity associated with a first regime of atrial fibrillation as well as any second or subsequent such map.

Those maps may be used to guide additional mapping and/or treatment. For example, during a cardiac mapping procedure, those maps may be displayed (e.g., on a monitor) allowing a clinician to decide in what areas of the heart to gather additional data. Similar, during an ablation procedure, because the potentially independent incidences of atrial fibrillation are drawn or shown on independent maps, a clinician may be able to identify a focal or driver or re-entry circuit associated with each incidence or regime of atrial fibrillation, providing the clinician with the necessary information about location to ablate to treat the condition.

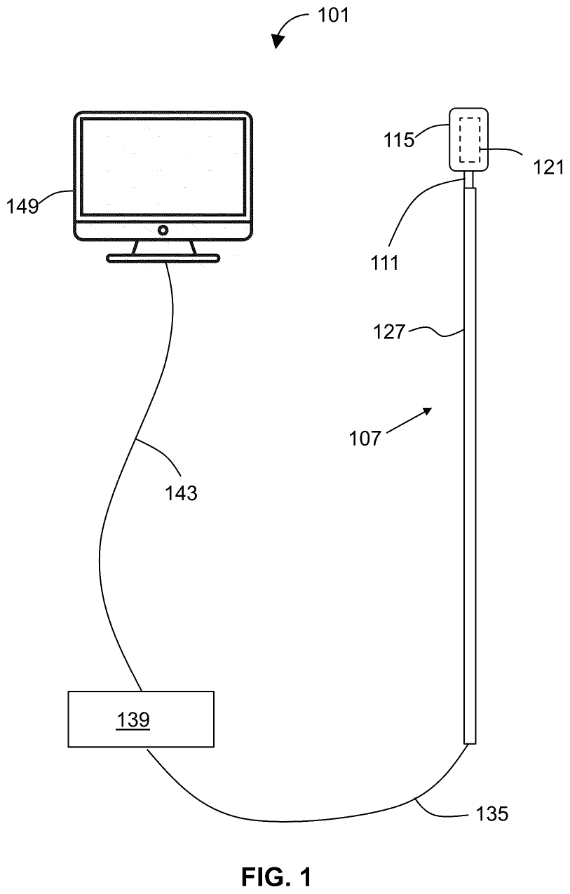

FIG. 1 shows a system 101 for mapping arrythmia or fibrillation and monitoring cardiac rhythm. The system 101 includes a cardiac mapping catheter 107 that may generally include a shaft 111 extending to a head 115 carrying an electrode array 121. Electrodes of the electrode array 121 may optionally be connected via leads or at least one signal wire 135 to an optional processing device 139, which in turn may have a data connection 143 to a computer system 149. The extended shaft 111 may extend through, and out of a distal end of, an introducer sheath 127. The computer system 149 may record signals detected by the electrodes of the catheter 107 and then analyze the signals to identify locations and incidences of atrial fibrillation in the heart. The computer system 149 has at least one processor coupled to a memory subsystem having instructions therein executable by the processor to cause the computer system 149 to organize measurements into one group with values within a first range and a second group with values within a second range and associate the first group with a first incidence of fibrillation and the second group with a second incidence of fibrillation in the heart.

The system 101 is useful to perform methods of the invention.

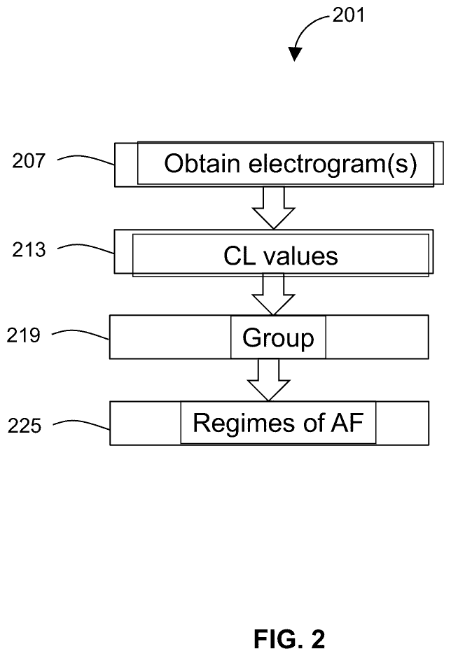

FIG. 2 shows steps of a method 201 of analyzing cardiac rhythm. The method 201 includes obtaining 207 measurements of electrical activity of a heart and measuring 213 components of certain values from those measurements. Preferably, the methods include measuring 213 cycle length (CL) values. Optionally, the method may include determining an average, e.g., median, for those values for example, within a sliding window (e.g., a 5 to 15 second sliding window over an approximately at least 3 to 30 minute time axis of an electrogram). Using a component such as the CL values measured in each position of the sliding window, the method 201 includes organizing 219 the measurements into one group with values within a first range and a second group with values within a second range. Having grouped 219 the segments of the electrograms (effectively having grouped beats that appear on the electrogram) into at least first and second groups, the computer system is operable to associate 225 the first group with a first incidence of fibrillation and the second group with a second incidence of fibrillation in the heart. It is noted that the first incidence of atrial fibrillation may be electrophysiological activity resulting from a first focal origin or multiple reentrant circuit and the second incidence of atrial fibrillation may be electrophysiological activity resulting from a second focal origin or multiple reentrant circuit potentially independent from the first focal origin or multiple reentrant circuit.

Methods of the invention use a mapping catheter to obtain 207 electrograms (shown and discussed in more detail below. Those measurements are used as inputs for the rhythm monitor method and system, by which portions of electrophysiological measurements are grouped by regime of atrial fibrillation. After the grouping by regime, methods may include subsequent data gathering which may also involve a cardiac catheterization.

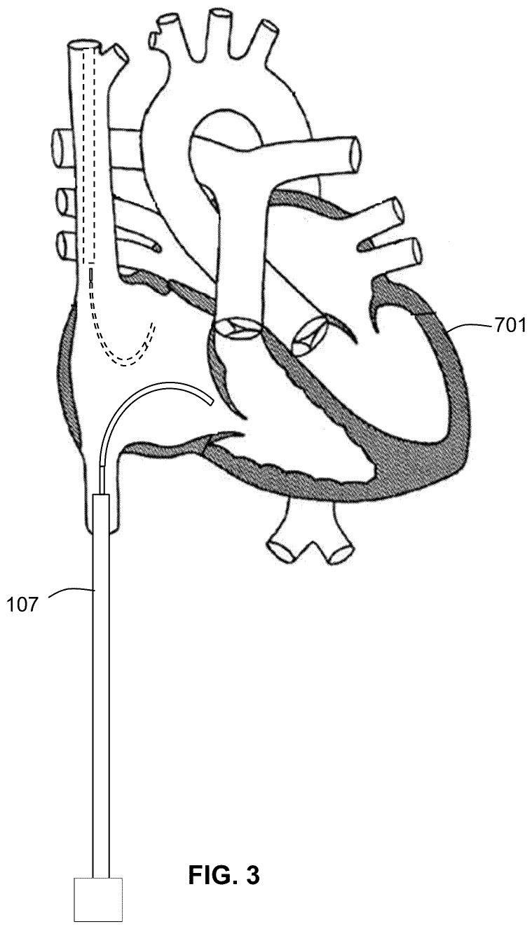

FIG. 3 shows obtaining the measurements of the electrical activity of a heart 701 by placing a catheter 107 of the invention into at least a coronary sinus (CS) of the heart 701 and creating a record of cycle length over a time between about a minute and about an hour. The catheter 107 may be advanced via the inferior vena cava (as drawn solid) or the superior vena cava (drawn in dashed lines). The catheter is operated, optionally held in one position, over a period of time such as a few minutes or about ten minutes or longer. Electrodes on the catheter gather data which may preferably be in the form of one or more electrogram(s) that may be stored and/or viewed on the computer system. Methods of the invention may involve analyzing those obtained measurements to determine cycle lengths (CL), e.g., for the beats measured by the electrograms. Systems and methods of the invention may then analyze that data via analysis of a record of CL over time.

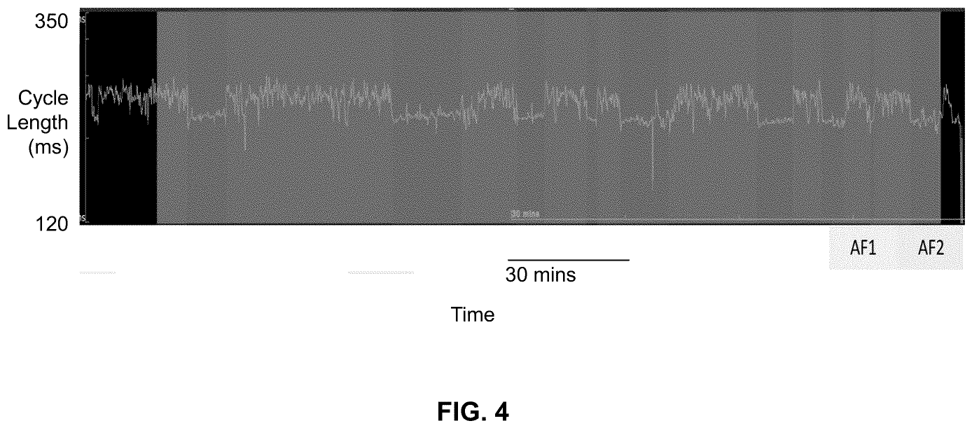

FIG. 4 shows a graph of cycle length (CL) over time as may be made and stored via systems and methods of the invention. Because some cardiac mapping procedures, e.g., using a CS catheter, acquire data sequentially, the resultant measurements may be subject to introduction of error via integrating sequentially acquired local information into a global whole (map) inappropriately when the global behavior has changed during the time of mapping. Using conventional approaches, there is no way to observe/detect global shifts in behavior. Using system and methods of the invention, electrogram behavior from one or more multi-electrode catheters that optionally do not move during mapping, is classified into stable regimes of behavior and changes from one stable regime to another are identified. Systems and methods of the invention are useful to segregate individual, sequential acquisitions (‘dwells’) into the appropriate maps (e.g., one for each stable regime). To segregate (or classify) the data, any suitable component(s) or dimensions of the electrogram data may be used. Certain embodiments classify the data based on: the CS catheter's CL median, variability and directionality. Those features may be grouped in 2D space. Each unique ‘space’ may be treated as a separate stable AF (e.g. ‘AF1’, ‘AF2’, etc.) and systems of the invention may deliver dwell data of those AF's into separate maps.

The graph shows cycle length (averaged across all 10 CS electrodes for one CS catheter embodiment) using a sliding window (between about 2 and 5 seconds) over time. The graph is not an electrogram but may be created by measuring, by the computer system, CLs in a sliding window over the time axis of an electrogram. The plot shows that cycle length varies with time (the line goes up and down very rapidly. Additionally, the plot goes up and down (slower and faster, respectively) around fairly stable means. Finally, it is evident that the plot varies around a lower or higher cycle length mean for minutes at a time and then abruptly switches to varying around a new mean. Those periods of time (indicated with shadings) represent two discrete “regimes” of “AF behavior”; i.e. the measured activity appears to follow one mean cycle length and then switch to a different cycle length, before switching back (and continuing to switch between those two CLs). Those two characteristic groupings of the CL measurements are taken to be manifestations of two potentially independent incidences of atrial fibrillation (AF), here referred to as (and labeled as) “AF1” and “AF2”. Systems and methods of the invention provide a rhythm monitor and are useful to detect those different states and assign any data gathered during a particular “AF” into a map that corresponds to that regime or incidence of AF.

The computer system 149 may perform any suitable analysis to group segments of the CL over time plot into distinct groupings. For example, in some embodiments, the computer system 149 uses a sliding window over a time dimension of the data to automatically determine cycle length medians within the sliding window and automatically group the portions (e.g., within the sliding window) of the electrograms into the two groups while the electrograms are being recorded. For the depicted plot, the sliding window was a few seconds (e.g., between two and five) and a coronary sinus (CS) catheter gathered the data for a few (e.g., about 3) hours. Preferably, the time dimension includes at least about five minutes of dwell time (e.g., 15 or 30 or 45 minutes or at least about an hour or 2 or 3) during which electrodes are performing the recording in the heart and optionally wherein the sliding window has a width between about 1 second and 10 seconds (e.g., about 2 second or 5 seconds) in the time dimension.

Systems and method of the invention group portions of the measurements by potentially independent incidences or regimes of atrial fibrillation. Systems and method of the invention may display activity of those potentially independent incidences or regimes of atrial fibrillation on separate maps. Once the regimes of AF have been identified, a mapping catheter may record additional data and systems here may determine which instance of atrial fibrillation the recorded data are associated with. Systems and method of the invention may display the new data, in real-time, on the appropriate cardiac map for that instance or area of fibrillation.

While discussed so far principally in terms of cycle length and cycle median, cycle length is one dimension by which systems and method of the invention group measurements and certain embodiments use a second or additional dimension of the measurements. For example, some embodiments use a measure of directionality as a dimension useful for grouping the data.

### Directionality

Embodiments of the invention include determining a direction of a wave of propagation in cardiac tissue. At least a component of a direction of propagation may be determined using a catheter with an array of electrodes. When electrophysiological data include directionality for waves wherein, methods may include identifying regimes of atrial fibrillation on the heart based on the direction of wave propagation across electrodes (as well as CL). Data from the heart that demonstrate similar directionality may be assigned to the same incidences of atrial fibrillation. Thus, methods may include grouping the measurements by the median cycle lengths and a second value, in which the second value comprises a direction of propagation. Any suitable approach may be used to measure a component of a direction of a wave. One approach makes use of a mapping catheter with an array of electrodes such that as a wave passes different electrodes of the array, the time of arrival at the different electrodes (and the known relative positions of those electrodes) may be used to construct at least a component of the direction.

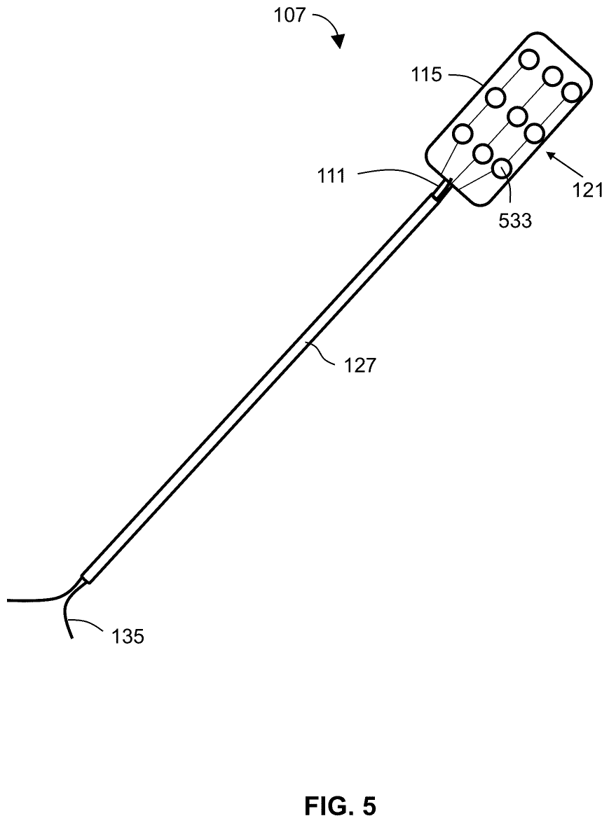

FIG. 5 gives a close-up view of the head 115 carrying an electrode array 121. The electrode array 121 may include a plurality of a sensing electrode 533 available on a tissue-facing surface of the head 115. The head 115 may include a shape memory and super-elastic material such as Nitinol, or other alloys of nickel and titanium such that when the head 115 within a sheath, it is substantially s-shaped, rolled, or folded. After the electrode array is deployed from a sheath, the head 115 opens to an unfurled configuration such that the electrode array 121 may lie against or near the tissue at which it is deployed. Optionally, the electrodes may be in a linear array, e.g., along a segment of catheter, such as on a coronary sinus (CS) catheter.

As shown, the cardiac mapping catheter 107 comprising an extended catheter body 111 dimension for percutaneous insertion into a chamber of the heart, and a deformable head 115 on a distal portion of the catheter body, with the electrodes disposed in an array over an area of the deformable head. In preferred embodiments, the catheter comprises an array of electrodes. More than one array of electrodes may be placed in the heart at any time, and the arrays may remain in a fixed position throughout the duration of the procedure. Each electrode may include electrode contact elements shaped substantially as thin discs disposed in array (e.g., rectangular grid; hexagonal pattern; spaced along set of a roughly parallel lines; or along a linear segment).

In non-limiting embodiments, a diameter or lateral extent of each electrode may be 150 μm such that the electrode surface area is less than or equal to 0.018 mm². The electrode array may comprise a number (e.g., 30, 40, 44, 50, 56 . . . ) of electrodes. The electrode array width may be less than or equal to 15 mm, with an overall length less than or equal to 26 mm. The array sensing area may be less than or equal to 15 mm² . The cardiac mapping catheter may be used with a sheath, such as an 8.5 F. steerable sheath with a diameter ranging from about 0.122 inches to about 2.83 mm.

Electrodes may be unipolar, bipolar, or of a hybrid arrangement involving a stacked pair of bipolar electrodes that provides advantages of unipolar electrodes. For conventional electrophysiological procedures, intra-cardiac electrodes have been deployed in two distinct arrangements, unipolar and bipolar. For bipolar recordings, a different electrode is positioned at the site of interest while the indifferent electrode is positioned at a separate site. Wave fronts that pass the electrodes induce similar signals at both poles, but with the signals being shifted in time. In the unipolar recording mode, a different electrode is positioned at the site where the electrical potential must be determined while the indifferent electrode is positioned a large distance from the heart, at zero potential. Unipolar electrodes are adequate but have a tendency to include far field electrical activity in the recorded signal which can result in a fractionated electrogram, of concern when trying to map complex arrhythmias (e.g. cardiac fibrillation) and accurately identifying local activation time. Bipolar electrode arrangements minimize that fractionation by placing both electrodes within the heart at a relatively narrow distance apart. Both electrodes of a bipolar pair measure approximately the same far field electrical activity with the result that the resultant electrogram includes little far field signal. Here, catheters of the disclosure may use another electrode arrangement comprising pairs of electrodes that are, in fact, bipolar but in which the pair is designed to sit in a stack over one location of the tissue so that, like with unipolar electrodes, signal recorded by the stacked electrode pair tends to reflect electrical activity from all regions of the heart even when the stacked electrode pair is placed directly over a particular region. Thus, each electrode may be provided as, or as part of, an electrode pair having a “stacked” or orthogonal close unipolar (OCU) electrode arrangement. This electrode array may include a two dimensional array (two dimensions in the plane of the page of FIG. 5) of stacked electrode pairs (e.g., first electrode and second electrode layered with insulator therebetween). The first electrode is configured to be close to a location on a surface of target tissue and the second electrode is separated from the first electrode but over the same location. Each electrode pair may be provided in an orthogonal, close, unipolar (OCU) arrangement. Of the pair, the two electrodes are stacked such that when the array is against tissue, an axis with rotational symmetry through the stack of the two electrodes is substantially orthogonal to a surface of the tissue where the electrode pair is making a reading (“orthogonal” nature of the pair). A distance between the first and second electrodes is preferably about a same order of magnitude as a diameter or thickness of each electrodes so that the members of the pair are close to each other (“close” nature of the pair). The one pair of bipolar electrodes may operate to measure electrical activity from all regions of the heart as expected from unipolar electrodes (“unipolar” nature of the pair).

Such an arrangement, referred to as an OCU arrangement, addresses the limitations of existing unipolar and bipolar electrodes. In particular, recorded electrical potential of current bipolar electrodes vary with their orientation relative to the direction of a passing wavefront. Additionally, because bipolar electrodes have both electrodes on a given surface, there is potential inclusion of distinctly different electrical activity from each electrode. As such, by providing electrodes oriented perpendicular to the tissue plane, via the orthogonal close unipolar (OCU) design of the present invention, the electrode array of the present disclosure retains the superior near/far-field discrimination of common bipolar electrode recordings with the directional independence and smaller footprint of unipolar recordings. Furthermore, the unipolar electrode arrangement of the present invention retains all of the spatial resolution benefits of a contact bipolar arrangement, but with the additional spatial resolution enhancement conferred by a smaller footprint.

For delivery by e.g., a percutaneous procedure, the head 115 may be substantially furled (or curled) closed, e.g., in a cylindrical configuration when the head is retracted within a sheath. When the head is extended out, in a distal direction, from a distal end of the sheath, the head 115 may assume a substantially flat, but deformable, aspect. Cardiac mapping catheters of the invention are capable of being reshaped when retracted or withdrawn into a sheath and unfurled when extended from a sheath. When held within a sheath 127, the electrode array 121 may be rolled or furled. When deployed from a sheath, the electrode array 121 unfurls and may lie at least partially against the tissue upon which it is deployed.

Catheters of the invention may include one or more location or position markers. Each position marker may be a magnetic coil, (e.g., as magnetic tracking sensors or magnetic location sensors). The position markers may be positioned on opposing sides and/or opposite ends of an array. Having magnetic coils in the opposing positions allows for shape detection of the array and identification of each electrode's position in 3D space. The position markers may provide a 5 or 6 degrees of freedom (DOF) sensor, wherein the degrees of freedom describe the number of axes in which a rigid body moves freely in 3D space. In some embodiments, data from the magnetic sensors are used to calculate the position of the electrode array to model a shape of a surface from which measurements are made.

In some embodiments, the electrode array may further comprise a plurality of pacing electrodes, positioned for the measurement of a refractory period and/or to stimulate activation of a cardiac rhythm. Thus, in some embodiments of the invention, the cardiac mapping catheters of the invention allow for sensing, mapping, and pacing functions on the single device.

In preferred embodiments, the electrodes remain in a fixed position throughout the duration of the procedure. Additionally, more than one electrode array may be used at a time. Because a wave will propagate across multiple of the electrodes 533 and because their relative positioning is known, time data (e.g., arrival time) of the wave can be used to construct at least a component of a directionality of the wave.

In some embodiments, only one dimension of a direction of a wave is determined or used. For example, the head 115 is positioned at a distal portion of the catheter 107. The electrodes 533 are arrayed over two dimensions that include a distal-to-proximal first dimension over the surface of the head 115 and a lateral second dimension that extends across the head 115 perpendicular to the first dimension. For each wave, a component of the direction in the first dimension may be determined and e.g., optionally normalized into a range from −1.0 to 1.0, in which −1.0 is entirely distal-to-proximal, zero is lateral, and 1.0 is proximal to distal.

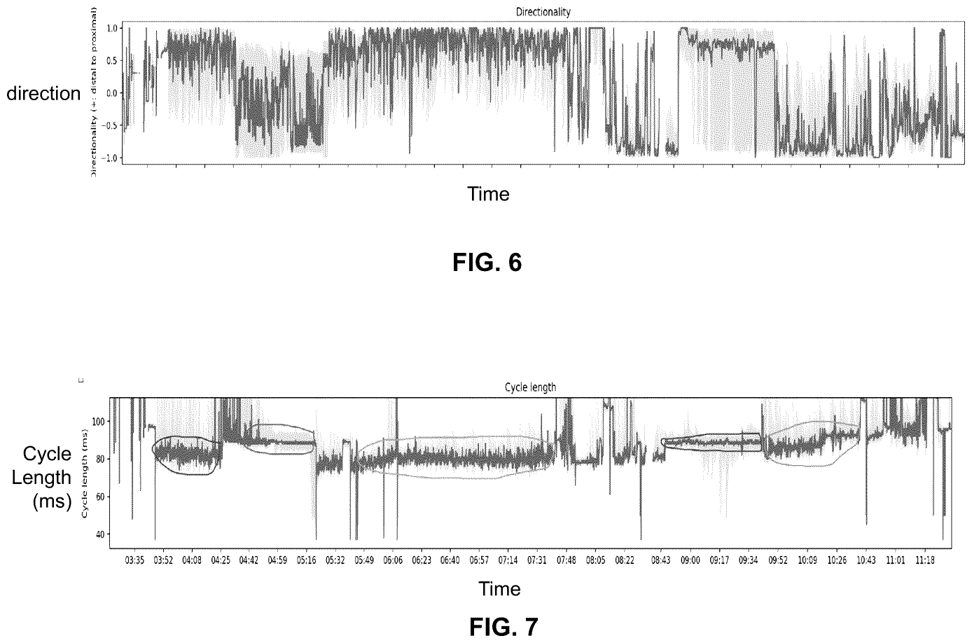

FIG. 6 shows a measurements of directionality of waves, over time, in the distal-to-proximal first dimension over the surface of the head 115 normalized into a range from −1.0 to 1.0. Visual inspection reveals groupings (by time) of waves that are tending to travel in consistent directions in the first dimension. The figure is a plot of CS directionality plotted against time.

FIG. 7 is a plot of CS cycle length over time (same axis labels as FIG. 4) and time axis corresponds to that for FIG. 6. In the plot, measurements appear to be grouped by CL median however, a directionality dimension from the directionality plot further distinguishes the groups. Portions of the plot have circles drawn to annotate sub-groups with common directionality there even where the directionality is distinct between groups (first, second, and third groups appear to have similar CL but second group (from plot above) has a directionality opposed to directionality of first and third groups).

Systems and methods of the invention may operate to group portions of the incoming data based on at least two dimensions including, for example, cycle length (e.g., specifically median CL within a 2 or 5 s sliding window) and directionality (e.g., in one dimension, scaled to −1.0 to 1.0). With the data appropriately collected, calculated, and stored in the computer system 149, any suitable operation may be performed to determine the distinct groups. For example, the computer system may implement a least squares operation or an analysis of variance operation to identify distinct groups that best segregate the windows of data.

Systems of the invention may further be able to display the depicted plots or even to display a combined plot showing the evidence of the potentially distinct regimes of atrial fibrillation.

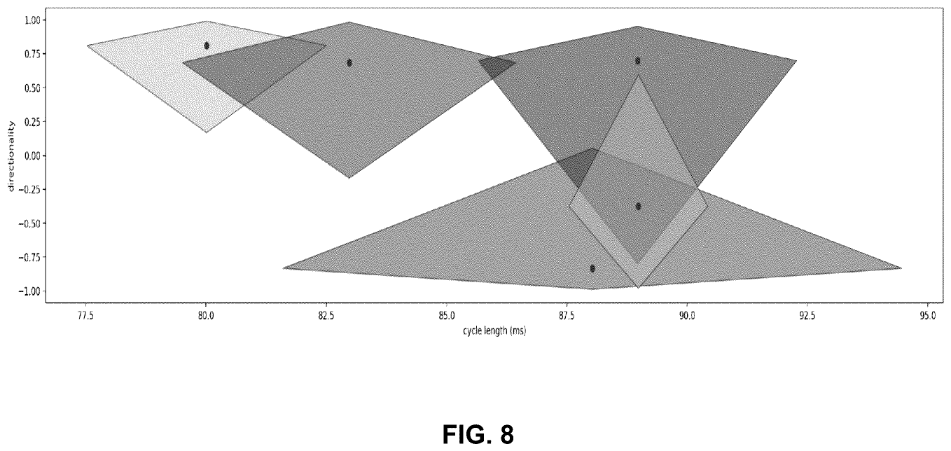

FIG. 8 displays the direction of activation along a CS catheter vs CS cycle length. Shading is used to show five “regimes” or incidence of atrial fibrillation. (Shading corresponds to shading used to call out, or circle, groups in the plot of CS cycle length over time.) In the depicted embodiments, a linear catheter was used, and directionality refers to where the electrodes on the distal end (of the tip) are excited before those on a proximal portion of the tip (or vice versa). The same directionality result could be obtained for a 2D mapping catheter such as the catheter 107. In the depicted plot there is a grouping of beats acquired in the CS. The drawn groups (the five bounding diamond-like shapes) surround those beats that share similar directionality AND similar cycle length. There is some overlap between groups (e.g. the two at the top left). As shown, the “regimes” may be particularly discernible in one or more of the “dimensions” measured (i.e. directionality or CL mean (one could also use CL variance as a 3rd dimension)).

Thus, methods may include identifying beats in the electrograms and assigning, to the at least two groups (here, 5), sets of the beats based on directionality and cycle length. Using the disclosed systems, the invention provides methods for mapping atrial fibrillation. Methods include recording electrograms from a heart using a plurality of electrodes disposed on a distal portion of a mapping catheter 107 (or a linear CS catheter) and storing the electrograms in memory of the computer system 149. The computer system 149 groups portions of the electrograms into at least two groups based at least on cycle length values determined from the electrograms and identifies an independent regime of atrial fibrillation in the heart for each of the at least two groups. The electrodes 522 may be disposed in an array over an area of a deformable head 115 at the distal portion of the mapping catheter 107. As shown, methods may include determining, from the electrograms and from time data for signals measured at the electrodes in the array over the area, at least a component of a direction of waves in the heart. In some embodiments, the cycle length values include median cycle length within a sliding window over different portion of the electrograms.

By performing the grouping and identifying distinct, and potentially independent, regimes or incidences of atrial fibrillation, systems and methods of the invention provide a mechanism by which to create separate maps to show those distinct, and potentially independent, regimes or incidences of atrial fibrillation and further to assign subsequent measurements to one of the maps and to update the maps with the subsequent measurements as those are made.

As the system analyzes the cardiac signals detected by the electrodes, a cardiac map representing a visual depiction of the data may be formed in real-time. For example, as the algorithm analyzes the cycle length median of the collected data, the algorithm may attribute certain signals gathered from specific locations in the heart to a specific instance of atrial fibrillation. Meanwhile, other signals that generate a different cycle length median value may be mapped onto a separate cardiac map. The computer may display one or more cardiac maps.

### CL Median

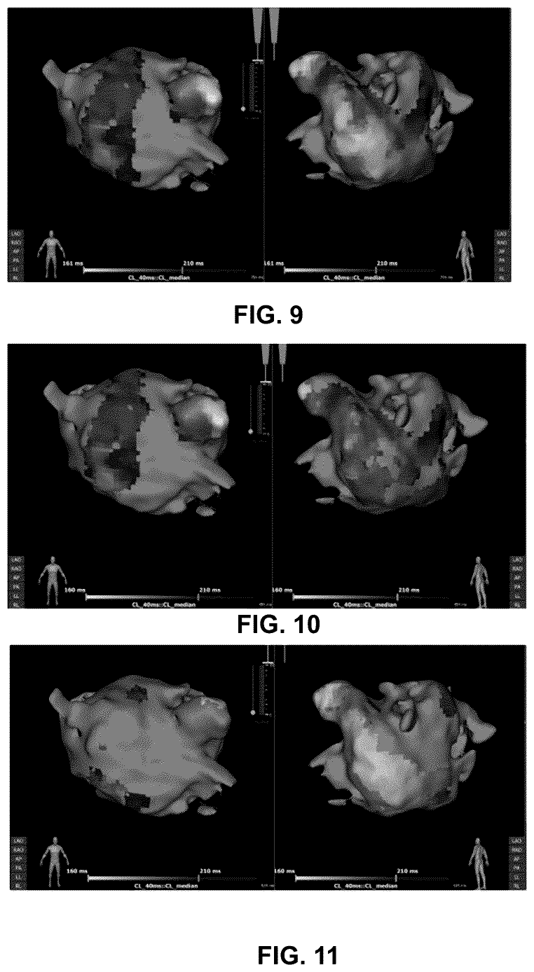

FIG. 9 shows a total (non-segregated) EP map showing electrophysiological activity in heart and specifically showing un-segregated CL median measurements mapped onto the heart using a color code (or a degree of shading) for the determined CL median. The left panel and the right panel show different positions of rotation in the Z direction of the same 3D digital map. The two panels show all of the cycle length medians as if those were aspects of a single phenomenon of interest. In the images, there is little basis by which to distinguish which areas correspond to which incidences or regimes of atrial fibrillation. The computer system may operate to store the first group and the second group in the memory and provide a report using the first group and second group to show the first incidence and the second incidence as manifestations of independent physiological causes of atrial fibrillation. That is, methods of the invention group those CL median measurements into two groups and create at least a first map showing activity of a first regime of AF in the heart and a second map showing activity of a second regime of AF in the heart.

FIG. 10 shows a first map showing CL median of a first regime of AF in the heart.

FIG. 11 shows a second map showing CL median of a second regime of AF in the heart.

Systems may include a display device in a surgical theater operable to display at least the first map or the second map. That is, the first and second map may be displayed on a monitor while a clinician performs a procedure. In other embodiments of the invention, the algorithm may use the signals recorded by the electrodes to calculate cycle length variance (CL variance) and display a cardiac map of CL variance. Here, the algorithm determines the cycle length mean as described earlier and then calculates the variance of each.

### CL Variance

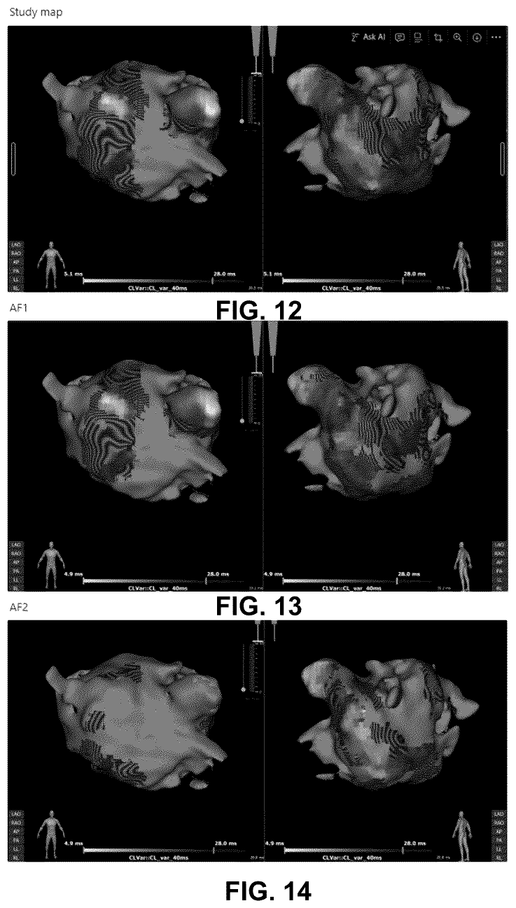

FIG. 12 shows an unsegregated map of cycle length variance on a 3D model of a heart. The left and right panels are images of the same 3D model and map at different rotational positions. Systems and methods of the invention operate similarly as described for CL median, but use variance of CL as a dimension of information for the grouping, e.g., to identify at least a first group and a second group arising for potentially independent incidences or regimes of atrial fibrillation. The system then uses those groups to segregate the shown, mapped activity into independent first and second maps of CL variance shown on a 3D model of heart.

FIG. 13 shows a first map of cycle length variance on a 3D model of a heart for a first regime of atrial fibrillation (AF1).

FIG. 14 shows a second map of cycle length variance on a 3D model of a heart for second regime of atrial fibrillation (AF2).

### Prominence

Systems and methods of the invention may use a prominence mapping operation to find, identify, or estimate one or more source loci in the heart for waves of electrophysiological (EP) activity. Such operations may be referred to as prominence mapping, an operation for determining an area of prominence from which EP waves emanate. Prominence mapping may operate by comparing analogous points from all measurements at electrodes (e.g., arrival times) in a pairwise manner to identify, of each pair, which point is earlier in time. The system can then trace back to the earliest measurement(s) and (using position indicators from the mapping catheter) determine a location of the earliest manifestation of (and implicitly the locus of origin of) the EP waves.

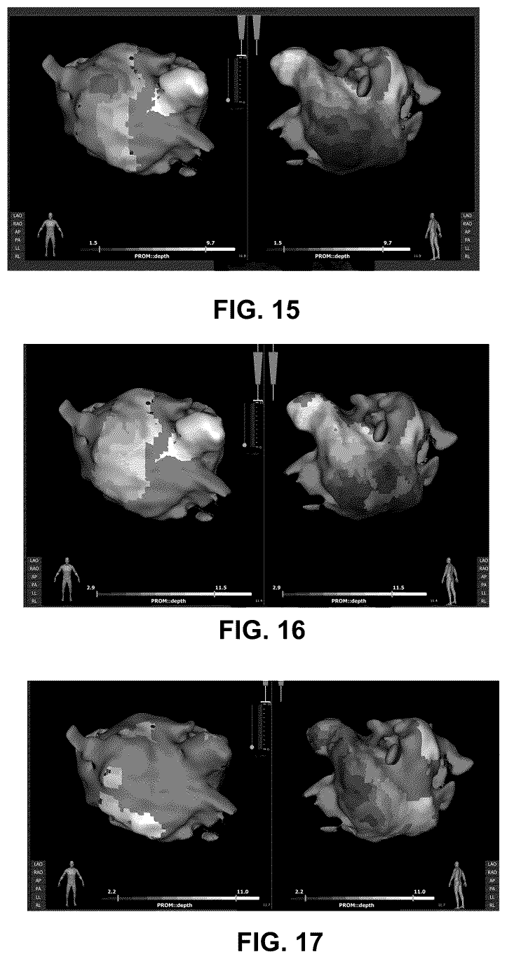

FIG. 15 shows an unsegregated prominence map depicted on a 3D representation of a heart. The map is an unsegregated map displaying all atrial fibrillation recorded by the electrodes after being subject to a prominence mapping operation. Using positional information, a 3D representation of the heart may be generated by the computer which displays the data graphically. The present invention provides methods and systems that use a cardiac mapping catheter and a “prominence mapping” algorithm to locate drivers of atrial fibrillation and thereby identify locations to ablate. Systems and methods of the invention use an array of electrodes to detect electrical activity in the heart. Each electrode records electrical activity of the heart as an electrogram. Each heartbeat will appear as a characteristic shape or signal across the electrograms. Each heartbeat involves a wave of electrical impulses that travels across the myocardium. As the wave passes an electrode, that wave appears as an event on the corresponding electrogram. Each electrogram is a measure of electrical activity at one specific location over time. Because each electrogram has a time axis, one wave will appear as events on two electrograms in the order in which the wave reached the corresponding electrodes. Methods and systems of the invention use a large number of electrodes to measure electrical activity in numerous places throughout the heart over time. Those measurements generate data in which cardiac electrical activity appears as a large number of events throughout the corresponding electrograms.

Those data are provided as input to a computer-implemented “prominence mapping” algorithm that will give, as output, a map or display that shows a location of the source of the aberrant electrical activity, a location referred as a driver of atrial fibrillation, or an AF driver. The prominence mapping algorithm of the invention makes comparisons among electrograms of time and location data for events to identify the direction of electrical waves and to trace a wave to a location at which that wave appeared. The regular electrical activity of the healthy heartbeat, originating at the sinoatrial node, is easily recognized as such (and ignored for the purpose of locating a stable driver of atrial fibrillation). Irregular activity characteristic of atrial fibrillation appears as originating from an AF driver. The prominence mapping algorithm may identify those locations by making pair-wise comparisons between the appearances of one wave on any two electrograms. Referencing the time component of the electrogram, the algorithm may identify which electrode the wave reached first. Using spatial positioning information from the electrodes, the algorithm may identify the specific location and direction of the wave through the cardiac tissue. The algorithm may iterate the pairwise comparisons over pairs of electrograms until the origin of a wave is located.

Methods may include measuring a plurality of electrograms (EGMs) from cardiac signals using electrodes on a catheter; processing the plurality of EGMs using the computer system to identify events in an EGM, wherein the event is stored in the computer; performing a prominence mapping operation to compare events obtained from different electrodes and that originate in an identical wave of activation to determine which event occurred first in time; assigning a positive point value to the event that occurred first in time; repeating the comparing and assigning steps until the plurality of EGMs have been compared to each other; and identifying a direction or origin of conduction based on the event with the highest positive point value. The event with the highest positive point value (or a local maxima) is indicative of a driver for atrial fibrillation.

However, the images in FIG. 15 are an un-segregated map and, if two more regimes of atrial fibrillation are occurring, a region of apparent prominence in the map may be imprecise. Methods may include analyzing a measurement (e.g., CL median and/or directionality) of the data and forming at least first and second groups of portions of the measurements, those groups corresponding to distinct and potentially independent regimes or incidences of atrial fibrillation.

The system can then create a first map showing a prominence mapping of a first regime of AF on a model of the heart and a second map showing a prominence mapping of a second regime of AF on the model of the heart. Local maxima in the prominence maps may indicate drivers of AF.

FIG. 16 shows a first prominence map depicted on a 3D representation of a heart for a first regime of atrial fibrillation (AF1).

FIG. 17 shows a second prominence map depicted on a 3D representation of the heart for a second regime of atrial fibrillation (AF2).

The computer system is optionally operable to annotate the first map and the second map (in any embodiment) to show evidence or a locus of the independent physiological causes as a focal driver or multi-wavelet re-entry circuit. For example, the system may draw a white dot or X or outline on a local maxima of prominence map, thereby indicating a likely locus of origination of fibrillogenic activity. It will be appreciated that methods and systems of the invention may involve displaying at least the first map during a cardiac mapping procedure to show areas in the heart where additional data should be collected or ablation could be performed.

Methods may include obtaining measurements of electrophysiological (EP) activity in the heart. In certain embodiments, methods include obtaining a plurality of cycle length measurements in a heart. Methods may include creating at least a first grouping of the cycle length measurements and a second grouping of the cycle length measurements, e.g., organizing the measurements into one group with values within a first range and a second group with values within a second range and correlating the first and second groups with respective first and second manifestations of atrial fibrillation in the heart. Here, “manifestation” is roughly synonymous with regime or incidence and those terms refer to sets of phenomena that are potentially associated with, or the outcome of, distinct and potentially independent physiological causes of atrial fibrillation. That is, each manifestation, regime, or incidence may be the result of a separate focal driver or multiple-wavelet reentrant circuit.

### Driver Identification

Systems and methods of the invention are useful for real-time cardiac signal analysis. Systems and methods of the invention use cardiac mapping catheters and/or ECG machines to measure cardiac signals. The measurements may be analyzed as they are acquired to identify, in real time, a start time and/or stop time of one or more drivers of cardiac fibrillation anywhere in the heart. The drivers and associated cardiac signals may be shown on the same or separate cardiac maps to provide a visual representation of electrical activity in the heart. Those maps may be used to guide additional mapping and/or treatment. For example, during a cardiac mapping procedure, the cardiac maps may be displayed (e.g., on a monitor that may be coupled to a computer system or machine collecting cardiac signals), to allow a clinician to decide in what areas of the heart to gather additional data. Similarly, during an ablation procedure, because the drivers of cardiac fibrillation and corresponding cardiac signals may be drawn or shown on independent cardiac maps, a clinician may be able to identify a focal driver or re-entry circuit associated with each driver of cardiac fibrillation, thereby providing the clinician with the location to ablate to treat the condition.

Methods and systems of the invention may use any of the catheters and electrode layouts described herein, such as cardiac mapping catheter 107 and electrode array 121. A computer system 149 may be coupled to a catheter or electrodes via leads or at least one signal wire 135. The computer system may be any computing system (hardware, firmware, software, or combinations thereof) operable to process the measured cardiac signals. In certain embodiments, the computer system includes memory and one or more processors able to execute methods of the invention. In preferred embodiments, the computer system analyzes the cardiac signals to identify driver start/stop time in “real time.” A driver of cardiac fibrillation refers to a source or feature on the heart that initiates or contributes to cardiac fibrillation. Examples include, but are not limited to, focal drivers, reentrant circuits, rotational activity, or other repeating activation features. Drivers may be intermittent, may migrate, and may overlap in time.

Systems and methods of the invention may identify a start time of a driver as the time or moment when the driver transitions into an active state. The stop time of a driver may be identified when the driver transitions into an inactive state. As used herein, “real time” includes identifying driver start/stop times while cardiac signals are being measured, such that outputs (e.g., current activity of a driver, driver start/stop times, or driver locations) can be produced or displayed with low latency to be clinically useful during mapping or ablation procedures.

In some embodiments, electrodes on the catheter may be positioned a distance away from a driver, and the computer system may still identify the start and/or stop time of that driver. Accordingly, systems and methods of the invention need not rely on direct contact with a driver of cardiac fibrillation to detect the driver's start or stop time.

Systems and methods of the invention may use the detected cardiac signals and drivers to create a cardiac map, as described previously. In certain embodiments, the cardiac map may be segmented based on the time period a driver was active. In situations where more than one driver is present, a cardiac signal may be categorized based on which drivers were active during the time period the cardiac signal was detected. The categorized signals may be mapped on separate cardiac maps in real time. In such embodiments, separate cardiac maps may represent different combinations of drivers (e.g., Driver A only, Driver B only, Drivers A+B, or no driver), thereby providing multiple maps that isolate electrical activity associated with each driver and combination thereof. When a cardiac map does not display a driver or displays a different driver at a different location, it may indicate the presence of an intermittent or missed driver.

Systems and methods of the invention may include an ECG machine coupled to the computer system 149. The ECG may provide additional information about global cardiac activity that may complement intracardiac measurements. The ECG may be a traditional 12-lead ECG where ten electrodes are placed on a person's limbs and on the surface of the chest. The overall magnitude of the heart's electrical potential is then measured from twelve different angles (“leads”) and is recorded over a period of time (usually ten seconds). In this way, the overall magnitude and direction of the heart's electrical depolarization is captured at each moment throughout the cardiac cycle.

There are three main components to an ECG: the P wave, which represents depolarization of the atria; the QRS complex, which represents depolarization of the ventricles; and the T wave, which represents repolarization of the ventricles. The traditional 12-lead placement is optimized for measuring ventricular signals. Therefore, certain embodiments of the invention use alternative lead placements to optimize detection of atrial signals. One alternative placement may include placing the V2, V4, and V6 leads on a person's back. Such placements may assist in identifying which chamber of the heart drivers of atrial fibrillation are located in. The placement may also provide information about the number of drivers in each chamber.

In certain embodiments, the alternatively placed ECG leads may be transformed into the frequency domain. For example, the ECG leads may be subject to a Fourier transform, a Fast Fourier Transform, or any other suitable transform. From the resulting frequency domain, the number of drivers in a chamber may be inferred from the number of prominent peaks and/or peak families, and chamber location may be inferred from relative amplitude of specific frequency peaks across different ECG leads (because different leads view cardiac activity from different angles). In certain embodiments, a peak family is defined as a cluster of peaks that remain within a frequency tolerance over time (e.g., within ±0.1 Hz, ±0.2 Hz, or ±0.5 Hz across a set of sliding windows).

Systems and methods of the invention may estimate how much each driver affects electrical activity in the chamber where that driver is located. In some embodiments, the effect may be quantified using one or more metrics such as the spectral power or amplitude of a driver-associated peak or band or the fraction of time the driver-associated peak is dominant. Other embodiments may determine a driver's effect on electrical activity based on visual inspection of a cardiac map. For example, a cardiac map may display (e.g., through highlighting or coloring) the area of the heart affected by a driver of fibrillation based on cardiac signals associated with the driver, as described earlier. In such cases, the effect of the driver may be relative to the amount of area highlighted/colored on the cardiac map (e.g., the more area that is highlighted, the greater the influence).

Systems and methods of the invention may determine the rate and/or variability of an individual driver. The rate may relate to a cycle length, a number of repeating activations per unit time, and/or a number of “start” events per unit time. In some embodiments, the computer system determines variability of an individual driver, including variability of rate (frequency or cycle length), variability of spectral amplitude, variability of mapped activity, and/or variability of start/stop timing. Variability may be summarized using statistical measures such as variance and standard deviation.

1. A method of analyzing cardiac rhythm, the method comprising:
obtaining measurements of electrical activity of a heart;
organizing the measurements into one group with values within a first range and a second group with values within a second range; and
associating the first group with a first incidence of fibrillation and the second group with a second incidence of fibrillation in the heart.

2. The method of claim 1, wherein the values comprise cycle lengths or median cycle lengths.

3. The method of claim 2, further comprising grouping the measurements by the median cycle lengths and a second value, wherein the second value comprises a direction of propagation.

4. The method of claim 1, further comprising using the first and second groups to assign subsequent measurements of cardiac activity to the first or second incidence of fibrillation.

5. The method of claim 1, wherein the first incidence of atrial fibrillation comprises electrical electrophysiological activity resulting from a first focal origin or multiple reentrant circuit and the second incidence of atrial fibrillation comprises electrophysiological activity resulting from a second focal origin or multiple reentrant circuit independent from the first focal origin or multiple reentrant circuit.

6. The method of claim 1, further comprising creating a first map of wave propagation of the first incidence of fibrillation and a second map of wave propagation of the second incidence of fibrillation.

7. The method of claim 6, further comprising displaying at least the first map or the second map on a display device of a computer system during a surgical procedure for ablation.

8. The method of claim 1, wherein the first incidence and the second incidence are manifestations of independent physiological causes of atrial fibrillation.

9. The method of claim 8, wherein each of the independent physiological causes comprises a focal driver or multi-wavelet re-entry circuit.

10. The method of claim 1, wherein obtaining the measurements of the electrical activity of the heart comprises placing a catheter comprising electrodes into at least a coronary sinus of the heart and creating a record of cycle length over a time between about 1 minute and 1 hour.

11. A system for analyzing cardiac rhythm, the system comprising:
a cardiac mapping catheter comprising electrodes operable to obtain measurements of electrical activity of a heart; and
a computer system communicably coupled to the cardiac mapping catheter, the computer system comprising at least once processor coupled to memory having instructions stored therein executable by the processor to cause the computer system to
organize the measurements into one group with values within a first range and a second group with values within a second range, and
associate the first group with a first incidence of fibrillation and the second group with a second incidence of fibrillation in the heart.

12. The system of claim 11, wherein the values comprise cycle lengths or median cycle lengths.

13. The system of claim 12, wherein the system groups the measurements by the median cycle lengths and a second value, wherein the second value comprises a direction of propagation.

14. The system of claim 11, wherein the computer system uses the first and second groups to assign subsequent measurements to the first or second incidence of fibrillation.

15. The system of claim 11, wherein the computer system is operable to create a first map of wave propagation of the first incidence of fibrillation and a second map of wave propagation of the second incidence of fibrillation.

16. The system of claim 15, further comprising a display device in a surgical theater operable to display at least the first map or the second map.

17. The system of claim 15, wherein the computer system stores the first group and the second group in the memory and provides a report using the first group and second group to show the first incidence and the second incidence as manifestations of independent physiological causes of atrial fibrillation.

18. The system of claim 17, further wherein the computer system is operable to annotate the first map and the second map to show each of the independent physiological causes as a focal driver or multi-wavelet re-entry circuit.

19. The system of claim 11, wherein obtaining the measurements of the electrical activity of the heart comprises placing a catheter comprising electrodes into at least a coronary sinus of the heart and creating a record of cycle length over a time between about 1 minute and 1 hour.

20. The system of claim 11, wherein the cardiac mapping catheter comprises an extended catheter body dimension for percutaneous insertion into a chamber of the heart, and a deformable head on a distal portion of the catheter body, with the electrodes disposed in an array over an area of the deformable head.

21-72. (canceled)
